(19) United States
(12) Patent Application Publication
(10) Pub. No.: US 2015/0065567 A1
(43) Pub. Date: Mar. 5, 2015

(54) 1-PHENYLALKANECARBOXYLIC ACID DERIVATIVES FOR THE TREATMENT OF ALZHEIMER'S DISEASE AND MULTIPLE SCLEROSIS

(71) Applicant: Chiesi Farmaceutici S.p.A., Parma (IT)

(72) Inventors: Bruno Imbimbo, Parma (IT); Marina Pizzi, Parma (IT); Daniel Chain, Parma (IT)

(73) Assignee: Chiesi Farmaceutici S.p.A., Parma (IT)

(21) Appl. No.: 14/460,821

(22) Filed: Aug. 15, 2014

Related U.S. Application Data
(60) Provisional application No. 61/871,077, filed on Aug. 28, 2013.

(51) Int. Cl. C07D 309/32; C07C 53/18; C07C 61/04

(52) U.S. Cl. CPC C07D 309/32; C07C 61/04; C07C 53/18
USPC 514/460; 514/572; 514/557

(57) ABSTRACT

Administration of certain 1-phenylalkanecarboxylic acid derivatives is useful for treating, preventing and/or reducing the risk of developing Alzheimer's Disease and multiple sclerosis.

### BACKGROUND OF THE INVENTION

1. Field of the Invention

The present invention relates to methods for treating, preventing, and/or reducing the risk of developing Alzheimer's Disease. The present invention also relates to methods for treating, preventing, and/or reducing the risk of developing multiple sclerosis.

2. Discussion of the Background

Alzheimer's disease (AD) is the most common form of dementia. The basic pathological abnormalities in AD brains are amyloid plaques, neurofibrillary tangles and neuronal loss. Amyloid plaques are composed of β-amyloid peptides (Aβ) that are proteolytically produced from amyloid precursor protein (APP). APP is initially cleaved by ∃-secretase to generate a 99-residue carboxy-terminal fragment (CTFβ or C99) that is subsequently cleaved by (-secretase to generate Aβ. Proteolysis by (-secretase is heterogeneous and generates several Aβ species of different lengths. The most abundant species is a 40-residue peptide (Aβ40). A 42-residue variant (Aβ42) is also formed and, although is much less abundant than Aβ40, is more prone to fibril formation and is the initially and predominantly deposited Aβ species in AD brains. Gamma-secretase is a complex of four different membrane proteins: presenilin (PS), nicastrin, anterior pharynx-defective (Aph-1), and presenilin enhancer 2 (Pen-2). Presenilins are of exceptional pathophysiological importance since more than 150 autosomal dominant point mutations are known in these proteins, all of which cause aggressive and early-onset AD. Many point mutations of APP and PS result in increased production of Aβ42 and according to the so called “amyloid hypothesis,” the oligomeric forms of Aβ42 are the main cause of neuronal death in AD (see Lesné S, et al., Nature, 440: 352-357 (2006) and Shankar G M, et al., Nat. Med., 14: 837-842 (2008), both of which are incorporated herein by reference in their entireties). Thus, inhibition or modulation of (-secretase appears to be a logical strategy to decrease Aβ42 accumulation in AD patients.

While a number of highly potent inhibitors of (-secretase have been identified (see Olson R E, et al., Curr. Top. Med. Chem., 8: 17-33 (2008), which is incorporated herein by reference in its entirety), serious concerns about their toxicity have been raised since γ-secretase can cleave several other membrane proteins other than APP (see Lleo A., Curr. Top. Med. Chem., 8: 9-16 (2008), which is incorporated herein by reference in its entirety), the most pharmacologically relevant being the Notch receptor. Indeed, γ-secretase inhibitors block proteolysis of Notch by inhibiting cleavage at site 3 (see Lewis H D, et al., Biochemistry, 42: 7580-7586 (2003), which is incorporated herein by reference in its entirety). Physiological cleavage of Notch leads to release of the Notch intracellular domain (NICD), a protein fragment that is translocated to the nucleus where it regulates transcription of target genes involved in cell development and in differentiation of adult self-renewing cells. The inhibitory effects of γ-secretase inhibitors on Notch activation in embryonic and fetal development may not be of concern for the treatment of AD patients. However, it is known that Notch signaling plays an important role in the ongoing differentiation processes of the immune system (see Maillard I, et al., Immunity, 19: 781-791 (2003), which is incorporated herein by reference in its entirety), gastrointestinal tract (see Stanger B Z, et al., Proc. Natl. Acad. Sci. USA, 102: 12443-12448 (2005), which is incorporated herein by reference in its entirety) and epidermal differentiation process (see Panelos J, et al., Cancer Biol. Ther., 8: 1986-1993 (2009), which is incorporated herein by reference in its entirety). Indeed, treatment of mice with γ-secretase inhibitors can cause severe gastrointestinal toxicity and compromise the proper maturation of B- and T-lymphocytes (see Searfoss G H, et al., J. Biol. Chem., 278: 46107-46116 (2003) and Wong G T, et al., J. Biol. Chem., 279: 12876-12882 (2004), both of which are incorporated herein by reference in their entireties).

Recently, in two large 21-month Phase 3 trials conducted in more than 2,600 AD patients, an increased rate of skin cancer with semagacestat, a potent γ-secretase inhibitor, compared to placebo has been observed (see Eli Lilly and Company (2010), Lilly halts development of semagacestat for Alzheimer's disease based on preliminary results of Phase III clinical trials. Press Release Aug. 17, 2010, which is incorporated herein by reference in its entirety). In addition, an interim analysis of these two Phase 3 trials showed that cognition and the ability to complete activities of daily living of patients treated with semagacestat worsened over time to a statistically significantly higher rate than those treated with placebo (see Eli Lilly and Company (2010), Lilly halts development of semagacestat for Alzheimer's disease based on preliminary results of Phase III clinical trials. Press Release Aug. 17, 2010, which is incorporated herein by reference in its entirety). The reasons for the detrimental cognitive and functional effects of semagacestat in AD patients are unclear. The drug-induced accumulation in the brain of the neurotoxic C-terminal fragment of APP (CTFβ or C99) resulting from the γ-secretase block (see Gitter B D, et al., Neurobiol, Aging, 25 (Suppl 2): S571 (2004), which is incorporated herein by reference in its entirety) and the tendency of the drug to increase brain (see Lanz T A, et al., J. Pharmacol. Exp. Ther., 319: 924-933 (2006), which is incorporated herein by reference in its entirety) and CSF (see Bateman R J, et al. Ann. Neurol., 66: 48-54 (2009), which is incorporated herein by reference in its entirety) levels of the neurotoxic Aβ42 peptide could play a role (see Imbimbo B P, et al., Curr. Opin. Investig. Drugs, 10: 721-730 (2009), which is incorporated herein by reference in its entirety). Indeed, semagacestat has been shown to decrease dendritic spine density in mice (see Bittner T, et al., J. Neurosci., 2009; 29: 10405-10409 (2009), which is incorporated herein by reference in its entirety). Thus, in order to get safe and effective anti-AD drugs, A∃42 may need to be selectively lowered without inducing abnormal accumulation of CTFβ and without affecting the proteolysis of Notch. This can be realized by modulating rather than inhibiting (-secretase.

The first compounds of this type are certain non-steroidal anti-inflammatory drugs (NSAIDs) that are capable of altering the cleavage properties of γ-secretase to lower the production of Aβ42 and increase the formation of a shorter 38-residue species (Aβ38) (see Weggen S, et al., Nature, 414: 212-216 (2001), which is incorporated herein by reference in its entirety). These compounds include ibuprofen, sulindac sulfide, indomethacin, flurbiprofen, and others. The ability of these NSAIDs to modulate Aβ production is not related to their inhibitory activity on cyclooxygenase (COX). Binding of these NSAIDs to APP is more efficient than to Notch and thus their effects on Notch processing are minor (see Kukar T, et al., Curr. Top. Med. Chem., 8: 47-53 (2008), which is incorporated herein by reference in its entirety). These NSAIDs are not very potent toward lowering Aγ production and structural modifications of these molecules have been proposed (see Peretto I, et al., Curr. Top. Med. Chem., 8: 38-46 (2008), which is incorporated herein by reference in its entirety).

Certain new γ-secretase modulators endowed with selective Aβ42-lowering properties but devoid of COX inhibitory activity, thus suitable for chronic use in AD patients, have been reported (see Peretto I, et al., J. Med. Chem., 48: 5705-5720 (2005), which is incorporated herein by reference in its entirety). Within this new chemical series, 1-(3′,4′-dichloro-2-fluorobiphenyl-4-yl)cyclopropanecarboxylic acid (CHF 5074) proved to be of major interest. In human neuroglioma cells over-expressing the Swedish mutated APP (H4swe), CHF 5074 preferentially lowers Aβ42 secretion with an IC₅₀ of 3.6 μM (see Imbimbo B P, et al., J. Pharmacol. Exp. Ther., 323: 822-830 (2007), which is incorporated herein by reference in its entirety). CHF 5074 does not display inhibitory activity on COX-1 and COX-2 enzymes when employed at concentrations up to 100 μM and 300 μM, respectively (see Imbimbo B P, et al. Pharmacol. Res., 55: 318-328 (2007), which is incorporated herein by reference in its entirety). At 5 μM, no effects were observed on Notch intracellular cleavage in human embryonic kidney 293swe cells (see Imbimbo B P, et al., J. Pharmacol. Exp. Ther., 323: 822-830 (2007), which is incorporated herein by reference in its entirety). At 100 μM, CHF 5074 does not alter the expression profile of several NICD-responsive genes (see Imbimbo B P, et al., Pharmacol. Res., 55: 318-328 (2007), which is incorporated herein by reference in its entirety). In mice and rats, CHF 5074 appears to be well absorbed orally (74% and 50%, respectively) and slowly eliminated from plasma (t_{1/2}=12 and 20 hours, respectively) (see Peretto I, et al., J. Med. Chem., 48: 5705-5720 (2005), which is incorporated herein by reference in its entirety). CHF 5074 brain levels represent 3-14% of the corresponding plasma concentrations. In guinea-pigs, CHF 5074 shows a prolonged plasma elimination half-life (t_{1/2}≈30 hours) after oral administration and a brain penetration of 3-9%. In dogs, the drug is quantitatively absorbed by oral route (80-100%) and is eliminated slowly from plasma (t_{1/2}≈24 hours). Brain to plasma drug levels range between 5 and 7%. The main metabolite of CHF 5074 appears to be the glucuronide conjugated derivative.

Pharmacological studies in transgenic mouse models of AD have shown that CHF 5074 is active from neuropathological and behavioral points of view at oral doses of 60 mg/kg/day (see Imbimbo B P, et al., J. Pharmacol. Exp. Ther., 323: 822-830 (2007); Imbimbo B P, et al., Br. J. Pharmacol., 159: 982-993 (2008); and Imbimbo B P, et al., J. Alzheimers Dis., 20: 159-173 (2010), all of which are incorporated herein by reference in their entireties). At this dose level, the drug appears to be well tolerated by mice even after prolonged treatment for 4-9 months.

Recently, it has been reported that variants of TREM2 cause susceptibility to late-onset Alzheimer's disease. In particular, substitution of arginine by hisitidine at residue 47, R47H, of the TREM2 protein has been reported to increase susceptibility of Alzheimer's disease. See T. Jonsson, et al., The New England Journal of Medicine, Nov. 14, 2012; and H. Neuman, et al., The New England Journal of Medicine, Nov. 14, 2012, both of which are incorporated herein by reference in their entireties.

It has also been reported that common variants in ABCA7, MS4A6A/MS4A4E, EPHA1, CD33, and CD2AP are associated with Alzheimer's disease. See P. Hollingworth, et al., Nat Genet., vol. 43(5), pp. 429-435 (2011), which is incorporated herein by reference in its entirety.

Multiple sclerosis, also known as disseminated sclerosis or encephalomyelitis disseminata, is an inflammatory disease in which the insulating covers of nerve cells in the brain and spinal cord are damaged. This damage disrupts the ability of parts of the nervous system to communicate, resulting in a wide range of signs and symptoms, including physical, mental, and sometimes psychiatric problems. MS takes several forms, with new symptoms either occurring in isolated attacks (relapsing forms) or building up over time (progressive forms). Between attacks, symptoms may go away completely; however, permanent neurological problems often occur, especially as the disease advances.

While the cause is not clear, the underlying mechanism is thought to be either destruction by the immune system or failure of the myelin-producing cells. Proposed causes for this include genetics and environmental factors such as infections. MS is usually diagnosed based on the presenting signs and symptoms and the results of supporting medical tests.

There is no known cure for multiple sclerosis. Treatments attempt to improve function after an attack and prevent new attacks. Medications used to treat MS while modestly effective can have adverse effects and be poorly tolerated. Many people pursue alternative treatments, despite a lack of evidence. The long-term outcome is difficult to predict; with good outcomes more often seen in women, those who develop the disease early in life, those with a relapsing course, and those who initially experienced few attacks. Life expectancy is 5 to 10 years lower than that of an unaffected population.

It has also bee reported that TREM2-transduced myeloid precursors mediate nervous tissue debris clearance and facilitate recovery in an animal model of multiple sclerosis. See K. Takahashi, et al., PLoS Medicine, vol. 4, pp 0675-0689 (2007), which is incorporated herein by reference in its entirety.

Thus, there remains a need for methods for treating, preventing, and/or reducing the risk of developing Alzheimer's Disease. There also remains a need for methods for treating, preventing, and/or reducing the risk of developing multiple sclerosis.

### SUMMARY OF THE INVENTION

Accordingly, it is one object of the present invention to provide novel methods for treating, preventing, and/or reducing the risk of developing Alzheimer's disease.

It is another object of the present invention to provide novel methods for treating, preventing, and/or reducing the risk of developing Alzheimer's disease in a subject which express a variant of TREM2.

It is another object of the present invention to provide novel methods for treating, preventing, and/or reducing the risk of developing Alzheimer's disease in a subject which express the [R47H] variant of TREM2.

It is another object of the present invention to provide novel methods for treating, preventing, and/or reducing the risk of developing Alzheimer's disease in a subject which express a variant of ABCA7, MS4A6A/MS4A4E, EPHA1, CD33, or CD2AP.

It is another object of the present invention to provide novel methods for treating, preventing, and/or reducing the risk of developing Alzheimer's disease in a subject which express a variant of CD33.

It is another object of the present invention to provide novel methods for treating, preventing, and/or reducing the risk of developing multiple sclerosis.

It is another object of the present invention to provide novel methods for treating, preventing, and/or reducing the risk of developing multiple sclerosis in a subject which express a variant of TREM2.

It is another object of the present invention to provide novel methods for treating, preventing, and/or reducing the risk of developing sclerosis disease in a subject which express the [R47H] variant of TREM2.

It is another object of the present invention to provide novel methods for treating, preventing, and/or reducing the risk of developing multiple sclerosis in a subject which express a variant of ABCA7, MS4A6A/MS4A4E, EPHA1, CD33, or CD2AP.

It is another object of the present invention to provide novel methods for treating, preventing, and/or reducing the risk of developing multiple sclerosis in a subject which express a variant of CD33.

These and other objects, which will become apparent during the following detailed description, have been achieved by the inventors' discovery that certain 1-phenylalkanecarboxylic acid compounds may be used for preventing and/or treating Cognitive Impairment, for preventing and/or treating Mild Cognitive Impairment, and for preventing and/or reducing the risk of developing Alzheimer's Disease in a cognitively normal subject.

Thus, the present invention provides:

(1) A method for treating, preventing, and/or reducing the risk of developing Alzheimer's disease, comprising administering, to a subject in need thereof, an effective amount of a compound of formula (I):

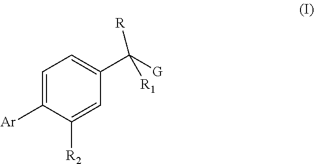

wherein:

R and R₁ are the same and are selected from the group of linear or branched C₁-C₄ alkyl; otherwise they form a 3 to 6 carbon atoms ring with the carbon atom to which they are linked;

G is: a COOR″ group wherein R″ is H, linear or branched C₁-C₄ alkyl, C₃-C₆ cycloalkyl or ascorbyl; a CONH₂ or a CONHSO₂R′″ group wherein R′″ is linear or branched C₁-C₄ alkyl or C₃-C₆ cycloalkyl; a tetrazolyl residue;

R₂ is H, CF₃, OCF₃ or a halogen selected from the group of F, Cl, Br, I, preferably fluorine. Ar is a group of formula

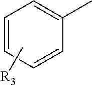

wherein R₃ represents one or more groups independently selected from:
halogen as previously defined;
—CF₃;
C₃-C₈ cycloalkyl optionally substituted with one or more C₁-C₄ alkyl and/or oxo groups;
—CH═CH₂;
—CN;
—CH₂OH;
methylendioxy or ethylendioxy;
—NO₂;
phenyl optionally substituted with one or more of the following groups:
   halogen;
   —CF₃;
   —OCF₃;
   —OH;
   linear or branched C₁-C₄ alkyl;
   a saturated heterocycle with at least 4 carbon atoms and at least 1 heteroatom;
   C₃-C₈ cycloalkyl in turn optionally substituted with one or more of the following groups linear or branched C₁-C₄ alkyl, CF₃ or OH;
—OR₄ or —NHCOR₄ wherein R₄ is CF₃, linear or branched C₂-C₆ alkenyl or alkynyl; benzyl; phenyl optionally substituted with one or more of the following groups: halogen, CF₃, OCF₃, OH, linear or branched C₁-C₄ alkyl; a saturated heterocycle with at least 4 carbon atoms and at least 1 heteroatom; C₃-C₈ cycloalkyl in turn optionally substituted with one or more of the following groups: linear or branched C₁-C₄ alkyl, CF₃ or OH;
SR₅, SO₂R₅ or COR₅ wherein R₅ is linear or branched C₁-C₆ alkyl; otherwise Ar is an optionally substituted heterocycle ring selected from the group of thiophene, benzothiophene, dibenzothiophene, thianthrene, pyrrole, pyrazole, furan, benzofuran, dibenzofuran, indole, isoindole, benzofurane, imidazole, benzoimidazole, oxazole, isoxazole, benzoxazole, thiazole, pyridine, pyrimidine, pyrazine, pyridazine, quinoline, isoquinoline, quinazoline, quinoxaline, cinnoline, pyrazole, pyran, benzopyran, pyrrolizine, phtalazine, 1,5-naphthyridine, 1,3-dioxole, 1,3-benzodioxole, optionally substituted with one or more groups R₃ as defined above; pharmaceutically acceptable salts and esters thereof.

(2) A method for treating, preventing, and/or reducing the risk of developing Alzheimer's disease, comprising administering, to a subject in need thereof, an effective amount of compound selected from the group consisting of:
1-(2-fluoro-4′-trifluoromethylbiphenyl-4-yl)cyclopropanecarboxylic acid;
1-(2-fluoro-3′-trifluoromethoxybiphenyl-4-yl)cyclopropanecarboxylic acid;
1-(2-fluoro-4′-trifluoromethoxybiphenyl-4-yl)cyclopropanecarboxylic acid;
1-(2-fluoro-3′-trifluoromethylbiphenyl-4-yl)-cyclopropanecarboxylic acid;
1-[2-fluoro-4′-(tetrahydro-pyran-4-yloxy)-biphenyl-4-yl]-cyclopropanecarboxylic acid;
1-(2,3′,4′-trifluorobiphenyl-4-yl)cyclopropanecarboxylic acid;
1-(3′,4′-dichloro-2-fluorobiphenyl-4-yl)cyclopropanecarboxylic acid;
1-(3′-chloro-2,4′-difluorobiphenyl-4-yl)cyclopropanecarboxylic acid;
1-[2-fluoro-4′-(4-oxo-cyclohexyl)-biphenyl-4-yl]cyclopropanecarboxylic acid;
2-(2″-fluoro-4-hydroxy-[1,1′; 4′,1′]terphenyl-4″-yl)propionic acid;
1-(2,2′,4″-trifluoro[1,1′; 4′,1″]terphenyl-4-yl)cyclopropanecarboxylic acid;
1-(2′,2″-difluoro-4-hydroxy[1,1′; 4′,1″]terphenyl-4″-yl)cyclopropanecarboxylic acid;
1-(2,2′-difluoro-4″-hydroxy[1,1′; 4″,1″]terphenyl-4-yl)cyclopropanecarboxylic acid; and

pharmaceutically acceptable salts thereof.

(3) A method for treating, preventing, and/or reducing the risk of developing Alzheimer's disease, comprising administering, to a subject in need thereof, an effective amount of 1-(3′,4′-dichloro-2-fluorobiphenyl-4-yl)cyclopropanecarboxylic acid or a pharmaceutically acceptable salt thereof

(4) A method according to any of (1)-(3), wherein said subject has been identified as being at risk of developing Alzheimer's disease.

(5) A method according to any of (1)-(3), wherein said subject has been identified as expressing a variant of TREM2.

(6) A method according to any of (1)-(3), wherein said subject has been identified as expressing the R47H variant of TREM2.

(7) A method according to any of (1)-(3), wherein said subject has been identified as expressing a variant of ABCA7, MS4A6A/MS4A4E, EPHA1, CD33, or CD2AP.

(8) A method according to any of (1)-(3), wherein said subject has been identified as expressing a variant of CD33.

(9) A method for treating, preventing, and/or reducing the risk of developing multiple sclerosis, comprising administering, to a subject in need thereof, an effective amount of a compound of formula (I):

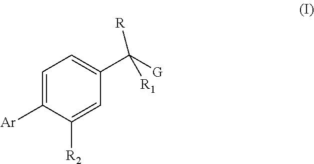

wherein:

R and R₁ are the same and are selected from the group of linear or branched C₁-C₄ alkyl; otherwise they form a 3 to 6 carbon atoms ring with the carbon atom to which they are linked;

G is: a COOR″ group wherein R″ is H, linear or branched C₁-C₄ alkyl, C₃-C₆ cycloalkyl or ascorbyl; a CONH₂ or a CONHSO₂R′″ group wherein R′″ is linear or branched C₁-C₄ alkyl or C₃-C₆ cycloalkyl; a tetrazolyl residue; R₂ is H, CF₃, OCF₃ or a halogen selected from the group of F, Cl, Br, I, preferably fluorine. Ar is a group of formula

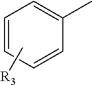

wherein R₃ represents one or more groups independently selected from:
halogen as previously defined;
—CF₃;
C₃-C₈ cycloalkyl optionally substituted with one or more C₁-C₄ alkyl and/or oxo groups;
—CH═CH₂;
—CN;
—CH₂OH;
methylendioxy or ethylendioxy;
—NO₂;
phenyl optionally substituted with one or more of the following groups:
   halogen;
   —CF₃;
   —OCF₃;
   —OH;
   linear or branched C₁-C₄ alkyl;
   a saturated heterocycle with at least 4 carbon atoms and at least 1 heteroatom;
   C₃-C₈ cycloalkyl in turn optionally substituted with one or more of the following groups linear or branched C₁-C₄ alkyl, CF₃ or OH;
—OR₄ or —NHCOR₄ wherein R₄ is CF₃, linear or branched C₂-C₆ alkenyl or alkynyl; benzyl; phenyl optionally substituted with one or more of the following groups: halogen, CF₃, OCF₃, OH, linear or branched C₁-C₄ alkyl; a saturated heterocycle with at least 4 carbon atoms and at least 1 heteroatom; C₃-C₈ cycloalkyl in turn optionally substituted with one or more of the following groups: linear or branched C₁-C₄ alkyl, CF₃ or OH;
SR₅, SO₂R₅ or COR₅ wherein R₅ is linear or branched C₁-C₆ alkyl; otherwise Ar is an optionally substituted heterocycle ring selected from the group of thiophene, benzothiophene, dibenzothiophene, thianthrene, pyrrole, pyrazole, furan, benzofuran, dibenzofuran, indole, isoindole, benzofurane, imidazole, benzoimidazole, oxazole, isoxazole, benzoxazole, thiazole, pyridine, pyrimidine, pyrazine, pyridazine, quinoline, isoquinoline, quinazoline, quinoxaline, cinnoline, pyrazole, pyran, benzopyran, pyrrolizine, phtalazine, 1,5-naphthyridine, 1,3-dioxole, 1,3-benzodioxole, optionally substituted with one or more groups R₃ as defined above; pharmaceutically acceptable salts and esters thereof

(10) A method for treating, preventing, and/or reducing the risk of developing multiple sclerosis, comprising administering, to a subject in need thereof, an effective amount of compound selected from the group consisting of:
1-(2-fluoro-4′-trifluoromethylbiphenyl-4-yl)cyclopropanecarboxylic acid;
1-(2-fluoro-3′-trifluoromethoxybiphenyl-4-yl)cyclopropanecarboxylic acid;
1-(2-fluoro-4′-trifluoromethoxybiphenyl-4-yl)cyclopropanecarboxylic acid;
1-(2-fluoro-3′-trifluoromethylbiphenyl-4-yl)-cyclopropanecarboxylic acid;
1-[2-fluoro-4′-(tetrahydro-pyran-4-yloxy)-biphenyl-4-yl]-cyclopropanecarboxylic acid;
1-(2,3′,4′-trifluorobiphenyl-4-yl)cyclopropanecarboxylic acid;
1-(3′,4′-dichloro-2-fluorobiphenyl-4-yl)cyclopropanecarboxylic acid;
1-(3′-chloro-2,4′-difluorobiphenyl-4-yl)cyclopropanecarboxylic acid;
1-[2-fluoro-4′-(4-oxo-cyclohexyl)-biphenyl-4-yl]cyclopropanecarboxylic acid;
2-(2″-fluoro-4-hydroxy-[1,1′; 4′,1′]terphenyl-4″-yl)propionic acid;
1-(2,2′,4″-trifluoro[1,1′; 4′,1″]terphenyl-4-yl)cyclopropanecarboxylic acid;
1-(2′,2″-difluoro-4-hydroxy[1,1′; 4′,1″]terphenyl-4″-yl)cyclopropanecarboxylic acid;
1-(2,2′-difluoro-4″-hydroxy[1,1′; 4″,1″]terphenyl-4-yl)cyclopropanecarboxylic acid; and

pharmaceutically acceptable salts thereof.

(11) A method for treating, preventing, and/or reducing the risk of developing multiple sclerosis disease, comprising administering, to a subject in need thereof, an effective amount of 1-(3′,4′-dichloro-2-fluorobiphenyl-4-yl)cyclopropanecarboxylic acid or a pharmaceutically acceptable salt thereof.

(12) A method according to any of (9)-(11), wherein said subject has been identified as expressing a variant of TREM2.

(13) A method according to any of (9)-(11), wherein said subject has been identified as expressing the R47H variant of TREM2.

(14) A method according to any of (9)-(11), wherein said subject has been identified as expressing a variant of ABCA7, MS4A6A/MS4A4E, EPHA1, CD33, or CD2AP.

(15) A method according to any of (9)-(11), wherein said subject has been identified as expressing a variant of CD33.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIGS. 1A, 1B, and 1C show the effects of 10 μM Aβ₁₋₄₂ on M1 (TNFα, IL-1β) and M2 (MRC1) markers in mixed glial cell culture. Quantification of TNFα (A), IL-1β (B) and MRC1 (C) expression levels in astrocyte-microglia co-culture exposed to Aβ₁₋₄₂ for 1, 2, 3 or 8 days measured by RT-qPCR. Exposure to Aβ₁₋₄₂ induces expression of the pro-inflammatory cytokines TNFα and IL-1β (markers of M1 microglia activation) and depresses expression of MRC1 (marker of M2 microglia activation). Changes in expression levels are maximal after 2 days of Aβ₁₋₄₂ exposure and fall down at 8 days.

FIGS. 2 A and 2B show the effects of 3 μM CHF5074 on M1 markers in mixed glial cell culture exposed to Aβ₁₋₄₂ 10 μM for 2 or 8 days. (A) Quantification of TNFα, IL 1β and iNOS expression levels in astrocyte-microglia co-culture exposed to Aβ₁₋₄₂ for 2 days analyzed by RT-qPCR. Two days exposure of astrocyte-microglia co-culture to 3 μM CHF5074 in the presence of 10 μM Aβ₁₋₄₂ reduces expression of pro-inflammatory cytokines TNFα, IL1β and iNOS (** p<0.01 *** p<0.001 vs corresponding vehicle; # p<0.05, ### p<0.001 vs corresponding Aβ). (B) Quantification of TNFα, IL1β and iNOS expression levels in astrocyte-microglia co-culture exposed to Aβ₁₋₄₂ for 8 days analyzed by RT-qPCR. CHF5074 does not modify the microglia inactivation state at 8 days.

FIGS. 3A and 3B show the effects of CHF5074 3 μM on M2 markers in mixed glial cell culture exposed to Aβ₁₋₄₂ 10 μM for 2 or 8 days. (A) Quantification of MRC1, TREM2 and ARG1 expression levels in astrocyte-microglia co-culture exposed to Aβ₁₋₄₂ for 2 days analyzed by qPCR. Two days exposure of astrocyte-microglia co-culture to 3 CHF5074 in the presence of 10 μM Aβ₁₋₄₂ increase expression of anti-inflammatory cytokines MRC1, TREM2 and ARG1 (** p<0.01 *** p<0.001 vs corresponding vehicle; # p<0.05, ### p<0.001 vs corresponding Aβ). (B) Quantification of MRC1, TREM2 and ARG1 expression levels in astrocyte-microglia co-culture exposed to Aβ₁₋₄₂ for 8 days analyzed by RT-qPCR. No changes in gene expression are observed in cultures exposed to Aβ₁₋₄₂ alone and in the presence of CHF5074 for 8 days.

### DETAILED DESCRIPTION OF THE PREFERRED EMBODIMENTS

Unless defined otherwise, all technical and scientific terms used herein have the same meaning as it is commonly understood by one of skill in the art to which this subject matter belongs.

All the terms “active drug,” ‘active ingredient,” “active,” “active substance,” “active compound,” and “therapeutic agent” are used synonymously.

As used herein, “an effective amount of a compound for treating a particular disease” is an amount that is sufficient to ameliorate, or in some manner reduce, the symptoms associated with the disease.

As used herein, the term “treatment” means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable.

As used herein the terms “prevention” and “preventing” mean reducing the occurrence of the disease, reducing the likelihood of contracting the disease, or delaying the onset of the disease.

With the term “APOE”, it is meant a class of apolipoproteins essential for the normal catabolism of triglyceride-rich lipoprotein constituents. ApoE is polymorphic with three major isoforms ApoE2, ApoE3, ApoE4. The form E4 has been implicated in impaired cognitive function.

In the case of non-amnestic MCI patients, criterium 2 of the original Petersen et al's criteria (see Petersen R C, et al., Arch. Neurol., 56: 303-308 (1999), which is incorporated herein by reference in its entirety) is also integrated with the diagnostic decision process suggested by Petersen (see Petersen R C, et al., J. Intern. Med., 256: 183-194 (2004), which is incorporated herein by reference in its entirety).

Thus, in a first embodiment, the present invention provides a method for treating, preventing, and/or reducing the risk of developing Alzheimer's disease, by administering, to a subject in need thereof, an effective amount of certain 1-phenylalkanecarboxylic acid compounds or a pharmaceutically acceptable salt thereof.

In a second embodiment, the present invention provides a method for treating, preventing, and/or reducing the risk of developing multiple sclerosis disease, by administering, to a subject in need thereof, an effective amount of certain 1-phenylalkanecarboxylic acid compounds or a pharmaceutically acceptable salt thereof.

With reference to formula (I), a first group of preferred compounds is that in which: R and R₁ form a 3 carbon atoms ring with the carbon atom to which they are linked;

R₂ is fluorine;

G is COOR″, wherein R″ is H, linear or branched C₁-C₄ alkyl, C₃-C₆ cycloalkyl or ascorbyl;

Ar is phenyl as defined above.

A second group of preferred compounds is that in which:

R and R₁ form a 3 carbon atoms ring with the carbon atom to which they are linked;

R₂ is fluorine;

G is CONH₂ or CONHSO₂R′″ wherein R′″ is linear or branched C₁-C₄ alkyl or C₃-C₆ cycloalkyl;

Ar is phenyl as defined above.

A third group of preferred compounds is that in which:

both R and R₁ are methyl;

R₂ is fluorine;

G is COOR″ wherein R″ is as defined above;

Ar is phenyl as defined above.

A fourth group of preferred compounds is that in which:

both R and R₁ are methyl;

R₂ is fluorine;

G is CONH₂ or CONHSO₂R′″, wherein R′″ is as defined above;

Ar is phenyl as defined above.

A fifth group of preferred compounds is that in which:

R and R₁ form a 3 carbon atoms ring with the carbon atom to which they are linked;

R₂ is fluorine;

G is COOR″ wherein R″ is as defined above;

Ar is a heterocycle as defined above.

A sixth group of preferred compounds is that in which: both R and R₁ are methyl;

R₂ is fluorine;

G is COOR″ wherein R″ is as defined above;

Ar is a heterocycle as defined above.

Particularly preferred are the following compounds:
2-methyl-2(2-fluoro-4′-trifluoromethylbiphen-4-yl)propionic acid (CHF 4810);
2-methyl-2(2-fluoro-4′cyclohexyl biphen-4-yl)propionic acid (CHF 4961);
1-(2-fluoro-4′-trifluoromethylbiphenyl-4-yl)cyclopropanecarboxylic acid (CHF 5022);
1-(4′-cyclohexyl-2-fluorobiphenyl-4-yl)cyclopropanecarboxylic acid (CHF 5023);
1-(4′-benzyloxy-2-fluorobiphenyl-4-yl)cyclopropanecarboxylic acid (CHF 5042);
1-(2-fluoro-4′-isopropyloxybiphenyl-4-yl)cyclopropanecarboxylic acid (CHF 5044);
1-(2-fluoro-3′-trifluoromethoxybiphenyl-4-yl)cyclopropanecarboxylic acid (CHF 5045);
1-(2-fluoro-4′-trifluoromethoxybiphenyl-4-yl)cyclopropanecarboxylic acid (CHF 5046);
1-(2-fluoro-3′-trifluoromethylbiphenyl-4-yl)cyclopropanecarboxylic acid (CHF 5058);
1-(4′-cyclopentyl-2-fluorobiphenyl-4-yl)cyclopropanecarboxylic acid (CHF 5059);
1-(4′-cycloheptyl-2-fluorobiphenyl-4-yl)cyclopropanecarboxylic acid (CHF 5060);
1-(2′-cyclohexyl-2-fluorobiphenyl-4-yl)cyclopropanecarboxylic acid (CHF 5061);
1-(2-fluoro-4′-hydroxybiphenyl-4-yl)cyclopropanecarboxylic acid (CHF 5070);
1-[2-fluoro-4′-(tetrahydropyran-4-yloxy)biphenyl-4-yl]-cyclopropanecarboxylic acid (CHF 5071);
1-(2,3′,4′-trifluorobiphenyl-4-yl)cyclopropanecarboxylic acid (CHF 5073);
1-(3′,4′-dichloro-2-fluorobiphenyl-4-yl)cyclopropanecarboxylic acid (CHF 5074);
1-(3′,5′-dichloro-2-fluorobiphenyl-4-yl)cyclopropanecarboxylic acid (CHF 5075);
1-(3′-chloro-2,4′-difluorobiphenyl-4-yl)cyclopropanecarboxylic acid (CHF 5076);
1-(4-benzo[b]thiophen-3-yl-3-fluorophenyl)cyclopropanecarboxylic acid (CHF 5077);
1-(2-fluoro-4′-prop-2-inyloxy-biphenyl-4-yl)-cyclopropanecarboxylic acid (CHF 5078);
1-(4′-cyclohexyloxy-2-fluoro-biphenyl-4-yl)-cyclopropanecarboxylic acid (CHF 5079);
1-[2-fluoro-4′-(tetrahydropyran-4-yl)-biphenyl-4-yl]-cyclopropanecarboxylic acid (CHF 5080);
1-[2-fluoro-4′-(4-oxo-cyclohexyl)-biphenyl-4-yl]-cyclopropanecarboxylic acid (CHF 5081);
2-(2″-fluoro-4-hydroxy-[1,1′:4′,1″]tert-phenyl-4″-yl)-cyclopropanecarboxylic acid (CHF 5083);
1-[4′-(4,4-dimethylcyclohexyl)-2-fluoro[1,1′-biphenyl]-4-yl]-cyclopropanecarboxylic acid (CHF 5084);
1-[2-fluoro-4′-[[4-(trifluoromethyl)benzoyl]ammino][1,1′-biphenyl]-4-yl]-cyclopropanecarboxylic acid (CHF 5094);
1-[2-fluoro-4′-[[4-(trifluoromethyl)cyclohexyl]oxy][1,1′-biphenyl]-4-yl]-cyclopropanecarboxylic acid (CHF 5096);
1-[2-fluoro-4′-[(3,3,5,5-tetramethylcyclohexyl)oxy][1,1′-biphenyl]-4-yl]-cyclopropanecarboxylic acid (CHF 5102);
1-[4′-[(4,4-dimethylcyclohexyl)oxy]-2-fluoro[1,1′-biphenyl]-4-yl]-cyclopropanecarboxylic acid (CHF 5103);
1-(2,3′,4″-trifluoro[1,1′:4′,1″-tert-phenyl]-4-yl)-cyclopropanecarboxylic acid (CHF 5104);
1-(2,2′,4″-trifluoro[1,1′:4′,1″-tert-phenyl]-4-yl)-cyclopropanecarboxylic acid (CHF 5105);
1-(2,3′-difluoro-4″-hydroxy[1,1′:4′,1″-tert-phenyl]-4-yl)-cyclopropanecarboxylic acid (CHF 5106);
1-(2,2′-difluoro-4″-hydroxy[1,1′:4′,1″-tert-phenyl]-4-yl)-cyclopropanecarboxylic acid (CHF 5107);
2-(2-fluoro-3′,5′-bis(chloro)biphen-4-yl)propionic acid amide (CHF 5125).

A more preferred group of compounds is that in which R and R₁ form a 3 carbon atoms ring with the carbon atom to which they are linked; R₂ is fluorine; G is COOH; Ar is phenyl substituted with one or more groups in such a way as that the log P (the partition coefficient between n-octanol and water) of the whole molecule is equal or higher than 4.5 as calculated in silico by using the software QikProp® release version 2.1 (Schrodinger Inc).

It has indeed been found that the higher the log P of the molecule, the greater is the inhibition potency of the release of Aβ₄₂ peptide and that particularly potent compounds are those whose log P is equal or higher than 4.5, preferably higher than 5.0.

The compounds and pharmaceutically acceptable salts thereof to be administered in the present invention are described and can be prepared as described in U.S. Pat. No. 7,662,995, which is incorporated herein by reference in its entirety.

In one preferred embodiment, the present methods comprise administration of 1-(3′,4′-dichloro-2-fluorobiphenyl-4-yl)cyclopropanecarboxylic acid or a pharmaceutically acceptable salt thereof, the compound of formula (Ia), also known with the internal code CHF 5074:

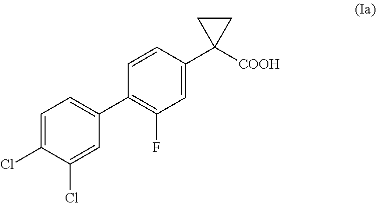 [SMILES: O=C(O)C1(c2ccc(-c3ccc(Cl)c(Cl)c3)c(F)c2)CC1]

In view of the close relationship between the compounds to be administered in the present methods, including the compound of formula (Ia), in the free acid form and those in the form of salts, the present invention is also directed to the use of pharmaceutically acceptable salts thereof. Suitable pharmaceutically acceptable salts according for use in the invention thus include those formed with both common organic and inorganic bases. For example, the salts disclosed in WO 2011/120778, which is incorporated herein by reference, may advantageously be utilized.

The compounds and pharmaceutically acceptable salts thereof to be administered in the present methods, including the compound of formula (Ia) and pharmaceutically acceptable salts thereof, may be combined with one or more pharmaceutically acceptable carriers or excipients to provide suitable pharmaceutical compositions. The pharmaceutically acceptable carriers or excipients may be advantageously selected from the group consisting of, diluents, wetting agents, emulsifying agents, binders, coatings, fillers, glidants, lubricants, disintegrants, preservatives, stabilizers, surfactants, pH buffering substances, flavouring agents, and the like. Comprehensive guidance on pharmaceutical excipients is given in Remington's Pharmaceutical Sciences Handbook, XVII Ed. Mack Pub., N.Y., U.S.A, which is incorporated herein by reference in its entirety.

The compounds and pharmaceutically acceptable salts thereof to be administered in the present methods, including the compound of formula (Ia) (CHF 5074) and pharmaceutically acceptable salts thereof, may be formulated for administration by any convenient route, e.g. by oral, parenteral, topical, inhalation, buccal, nasal, rectal, and transdermal administration. Suitable dosage forms can include tablets, capsules, lozenges, suppositories, solutions, emulsions, suspensions, syrups, ointments, creams, oils, and powders. Preferably, the pharmaceutical compositions of the present invention will be administered orally using appropriate dosage forms, such as capsules, tablets, caplets etc, more preferably tablets.

In one preferred embodiment, the compounds and pharmaceutically acceptable salts thereof to be administered in the present methods, including the compound of formula (Ia) (CHF 5074) and pharmaceutically acceptable salts thereof, will be administered in the form of tablets, comprising 200 mg of the active ingredient and the following excipients: lactose monohydrate, microcrystalline cellulose, hydroxypropylmethyl cellulose, croscaramellose sodium, sodium lauryl sulphate, and magnesium stearate.

However, it should be recognized that the dosage form may contain other amounts of the compound or pharmaceutically acceptable salt thereof, containing as little as 1 mg, 5 mg, 10 mg, 25 mg, 50 mg, 100 mg, 200 mg, 250 mg, 300 mg, 400 mg, 500 mg, or 1000 mg, and as much as 5 mg, 10 mg, 25 mg, 50 mg, 100 mg, 200 mg, 250 mg, 300 mg, 400 mg, 500 mg, 1000 mg, or 2000 mg, and any amount or range of amounts encompassed by these upper and lower amounts.

In one embodiment, the dosage of the compounds and pharmaceutically acceptable salts thereof to be administered in the present methods, including the compound of formula (Ia) (CHF 5074) and pharmaceutically acceptable salts thereof, and the duration of the treatment can vary within certain limits depending on the type of patient (weight, sex, subject age), the mode of administration and the severity advancement of the pathological condition or the specific memory or cognition disorder treated. A person skilled in the art may determine the optimal therapeutically effective amount and the regimen for each patient and thereby define the appropriate dosage and the duration of the treatment. For example, when the compounds and pharmaceutically acceptable salts thereof to be administered in the present methods, including the compound of formula (Ia) (CHF 5074) and pharmaceutically acceptable salts thereof, are administered by oral route to humans, a typical daily dosage might fall within the range of 100 mg and 800 mg, administered in a single or multiple daily dosage units, preferably between 200 and 600 mg.

In certain embodiments, the daily dose may be of 200 mg, in other embodiments, it may be of 400 mg, in further embodiments of 600 mg.

Of course, the daily dosage may range from as little as 1 mg, 5 mg, 10 mg, 25 mg, 50 mg, 100 mg, 200 mg, 250 mg, 300 mg, 400 mg, 500 mg, or 1000 mg, and as much as 5 mg, 10 mg, 25 mg, 50 mg, 100 mg, 200 mg, 250 mg, 300 mg, 400 mg, 500 mg, 1000 mg, or 2000 mg, and any amount or range of amounts encompassed by these upper and lower amounts.

The duration of the treatment may at least one year, but may be of a lesser or longer duration.

When the method is for the preventing or delaying the onset of Alzheimer's disease, the treatment may be commenced at any time and continued indefinitely. In some embodiments, the treatment may be commenced when the first signs of Alzheimer's disease are detected. In other embodiments, the treatment may be commenced when a subject has been identified asbeing at risk of developing Alzheimer's disease, such as being identified as expressing a variant of TREM2, such the R47H variant of TREM2.

In particular embodiments, the compounds and pharmaceutically acceptable salts thereof to be administered in the present methods, including the compound of formula (Ia) (CHF 5074) and pharmaceutically acceptable salts thereof, may be used in association with other therapeutic agents, for example with anticholinergic agents or with cholinesterase inhibitors and/or acetylcholine modulators.

The present invention provides a safe and highly effective therapeutic method, superior to existing treatments, for treating Alzheimer's disease.

In one embodiment of the invention, the subject being treated suffers from late-onset Alzheimer's disease or is at risk of developing late-onset Alzheimer's disease.

In another embodiment, the subject being treated expresses a variant of TREM2.

In another embodiment, the subject being treated expresses the R47H variant of TREM2.

In another embodiment, the subject being treated has been identified as expressing a variant of ABCA7, MS4A6A/MS4A4E, EPHA1, CD33, or CD2AP. In this context, the variant may be any which is associated with Alzheimer's disease. Examples of such variants are disclosed in P. Hollingworth, et al., Nat Genet., vol. 43(5), pp. 429-435 (2011), which is incorporated herein by reference in its entirety.

The subjects at the risk of developing Alzheimer's Disease may also carry the APOE4 gene and have a parental history of AD, more preferably they have an age between 45 and 65 years, two well-known independent risk factors of developing AD (see Debette S et al., Neurology, 73: 2071-2078 (2009), which is incorporated herein by reference in its entirety). The protective effects of NSAIDs on the AD onset may be restricted to APOE4 carriers (see Szekely et al., Neurology, 70: 17-24 (2008); Yip et al., NBMC Geriatrics, 5: 2 (2005); and Cornelius et al., Neuroepidemiology, 23: 135-43 (2004), all of which are incorporated herein by reference in their entireties). It has been shown that NSAIDs use may reduce the risk of AD onset in the middle age but at the same time NSAIDs use can accelerate the onset of AD in cognitively normal subjects with a median age of 75 years (see Breitner et al., Neurology, 72: 1899-1905 (2009), which is incorporated herein by reference in its entirety).

The present invention also provides a safe and highly effective therapeutic method, superior to existing treatments, for treating multiple sclerosis.

In one embodiment of the invention, the subject being treated is in recovery or tissue repair phase of multiple sclerosis.

In another embodiment, the subject being treated expresses a variant of TREM2.

In another embodiment, the subject being treated expresses the R47H variant of TREM2.

In another embodiment, the subject being treated has been identified as expressing a variant of ABCA7, MS4A6A/MS4A4E, EPHA1, CD33, or CD2AP. In this context, the variant may be any which is associated with Alzheimer's disease. Examples of such variants are disclosed in P. Hollingworth, et al., Nat Genet., vol. 43(5), pp. 429-435 (2011), which is incorporated herein by reference in its entirety.

Other features of the invention will become apparent in the course of the following descriptions of exemplary embodiments which are given for illustration of the invention and are not intended to be limiting thereof.

### EXAMPLES

### Methods.

### Primary Cell Culture.

C57BL/6 mice are purchased from Charles River. Mixed glial cell cultures were prepared from the brains of 1-3 day newborn mice. Briefly, after carefully removing meninges and large blood vessels, the brains were pooled and then dissociated by manual dispersion with a fire-polished Pasteur pipette. The cells were allowed to attach and grow at 37° C. in DMEM supplemented with 10% fetal bovine serum (FBS) (Euroclone), 4 mM L-glutamine, 200 U/ml penicillin/streptomycin in a water saturated atmosphere containing 5% CO₂ for two week.

### Beta-Amyloid Toxicity.

Soluble Aβ 1-42 were dissolved in sterile water to a final concentration of 2.5 mM, divided into aliquots and frozen. For experiments, mixed glial cells were incubated with soluble (not aggregated) Aβ 1-42, added at a concentration of 10 μM, in presence of CHF5074 (solved in 0.2% DMSO) or vehicle. Incubation was carried out for different times (from 1 to 8 days) in a Neurobasal/B27 medium. At the end of these periods, neuronal cell death by necrosis was evaluated by measuring the amount of lactate dehydrogenase (LDH) released into the culture medium using the CytoTox 96-non-radioactive cytotoxicity assay (Promega).

### Real-Time Reverse Transcription-Polymerase Chain Reaction (RT-PCR).

Total RNA was purified from cultured cells using the RNeasy Mini Kit for total RNA extractions (Qiagen). RNA was reverse transcribed using the Quantitect® Reverse Transcription Kit (Qiagen) with optimized mix of oligo-dT and random primers as primer and 1 μg of total RNA from mixed primary culture or co-culture. The reverse transcription reaction were performed in a total volume of 20 μL containing 5×RT-Buffer (that included dNTPs and Mg²⁺), 50 U RT (Reverse trascriptase), 20 U RNase Inhibitor in RNAse-DNase free water for 10 min at 25° C., 2 h at 37° C. and 10 min at 85° C. Individual 25 μL SYBR Green real-time PCR reactions consisted of 2 μL of cDNA, 12.5 μL of 2× iQ™ SYBR Green Supermix (Bio-Rad), and 1.5 μL of each 10 μM optimized forward and reverse primers in 7.5 μL RNase-free water.

The PCR were performed on a Bio-Rad iCycler Detection System using a 3-stage program: 3 min at 50° C., 10 min at 95° C. and 40 cycles of 30 sec at 94° C. and 45 secat 60° C. For standardization of quantification, β-actin were amplified simultaneously. Sets of primer were designed to amplify by Real-time RT-PCR (q-RT-PCR) and RT-PCR genes expressed by classically activated (M1) microglia:
TNFα (Tumor Necrosis Factor α);
IL-1β (Interleukin1β);
iNOS (inducible Nitric Oxide Sinthase);
   and by alternatively activated (M2) microglia:
ARG1 (Arginase 1);
MRC1 (C-type mannose receptor 1);
TREM2 (Triggering Receptor Expressed on Myeloid cells-2).
   Primer3plus was used as primer designing tool and then checked the oligos (synthetized by Invitrogen) to verify melting temperatures and quality of the sequences.

### Statistical Analysis.

Data were analyzed with Student's t-test for independent data. P<0.05 was considered as significant.

### Results.

Exposure of Astrocyte-Microglia Co-Culture to Aβ Induces Expression of Pro-Inflammatory. Cytokines in a Time-Dependent Manner

The capability of CHF5074 to modulate microglial activation in mixed culture of astrocyte/microglia exposed to beta-amyloid 42 (A13) was investigated.

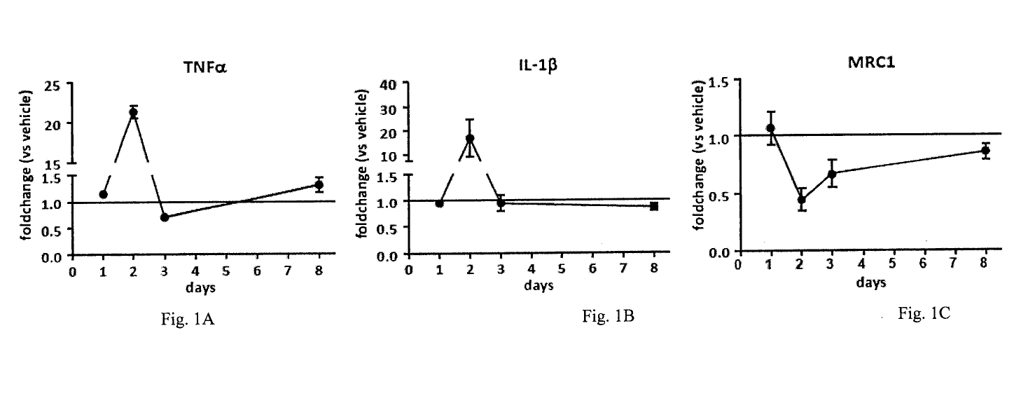

Astrocite-microglia co-cultures at 14° DIV were treated with a single dose of 10 μM soluble Aβ and the RNA was isolated at different time-points, i.e. 1 day, 2 days, 3 days, and 8 days later. After RNA retro-transcription to cDNA, quantitative RT-PCR analyses was applied to study the expression of pro-inflammatory cytokines tumor necrosis factor (TNFα) and interleukin-1β (IL-1β), as markers of M1 microglial activation, and mannose receptor type C-1 (MRC1) as a marker of phagocytic M2 microglia activation (see Martinez F O, et al., “Alternative activation of macrophages: an immunologic functional perspective,” Annu Rev Immunol., 2009; 27:451-483; Town T, et al., “The microglial “activation” continuum: from innate to adaptive responses,” J Neuroinflammation, 2005; 2:24; and Mosser D M, et al., “Exploring the full spectrum of macrophage activation,” Nat Rev Immunol., 2008 December; 8(12): 958-69, all of which are incorporated herein by reference in their entireties). No expression change was detected at 1 day, but a major inflammatory activation of microglia at 2 days from the Aβ addition was detected. The expression of TNFα and IL-1β mRNA markedly increased, while expression of MRC1 mRNA decreased. These changes were less evident 3 days after the Aβ addition and totally disappeared at the 8^{th} day of treatment (see FIG. 1).

### CHF5074 Reduces M1 Microglial Activation in Astrocyte-Microglia Co-Cultures Exposed to Aβ.

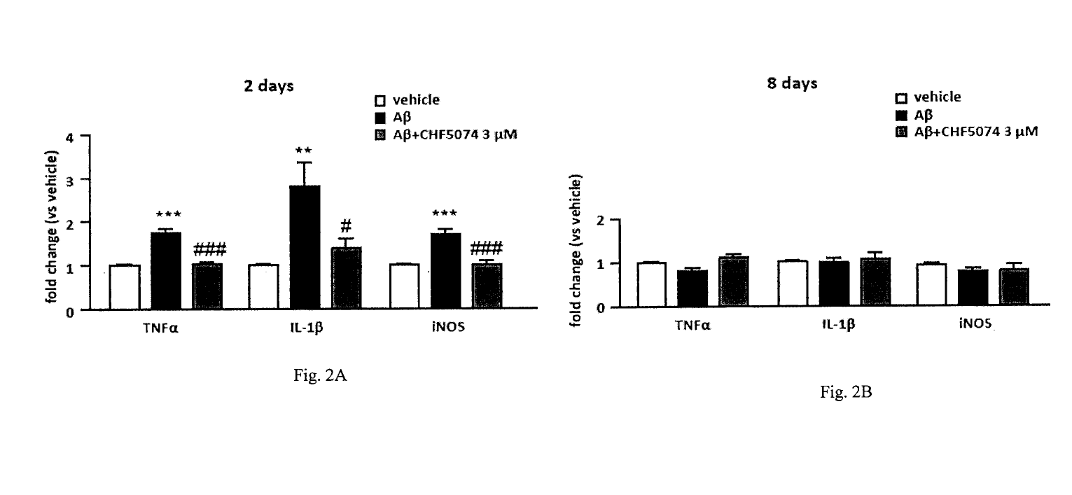

The effect of CHF5074 on the inflammatory response induced by Aβ was examined. Mixed astrocyte-microglia co-cultures were exposed to Aβ with or without CHF5074 at 3 μM concentration and examined for cytokine expression after 2 or 8 days. For classical activation of microglia, the expression of iNOS, beside TNFα and IL-1β was analyzed (see FIG. 2). The capability of Aβ to induce the inflammatory response in mixed glial cultures was confirmed, as the expression of iNOS as well as TNFα and IL-1β increased. Moreover, it was possible to detect anti-inflammatory effect of CHF5074, as the compound added simultaneously with Aβ significantly decreased either IL-1β or TNFα and iNOS mRNA expression (see FIG. 2A). After 8 days of Aβ treatment, expression of M1 genes fell to basal value and CHF5074 addition was unable to revert this effect (see FIG. 2B).

### CHF5074 Increases M2 Microglial Activation in Astrocyte-Microglia Co-Cultures Exposed to Aβ.

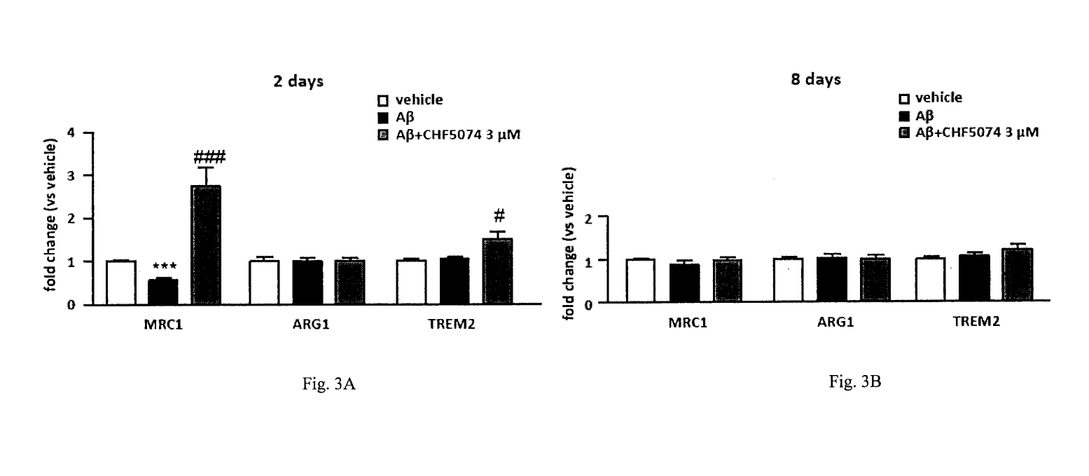

The alternative microglial activation markers (M2) in mixed glial cultures exposed to Aβ with and without CHF5074 for 2 days or 8 days were also investigated (see FIG. 3). The mRNA expression of MRC1 a cell surface receptor involved in antigen recognition and presentation and phagocytosis in macrophages (see Colton C A, et al., “Expression profiles for macrophage alternative activation genes in AD and in mouse models of AD,” J Neuroinflammation, 2006; 3:27, which is incorporated herein by reference in its entirety) and the triggering receptor expressed on myeloid cells-2 (TREM2), a member of the innate immune receptor TREM family, which induces cytoskeleton reorganization and increase phagocytosis (see Takahashi K, et al. “TREM2-trans-duced myeloid precursors mediate nervous tissue debris clearance and facilitate recovery in an animal model of multiple sclerosis,” PLoS Med 2007; 4(4):e124, which is incorporated herein by reference in its entirety) were checked. Arginase 1 (ARG 1), an additional M2 marker involved in mechanism of protection and repair of the extracellular matrix (see Colton C A, et al. “Expression profiles for macrophage alternative activation genes in AD and in mouse models of AD,” J Neuroinflammation, 2006; 3:27, which is incorporated herein by reference in its entirety), was also studied. Exposure to Aβ significant reduced the MRC1 expression to about 50% of basal value. This effect was reversed by CHF5074 which produced near a three-fold increase of MRC1 mRNA level. Moreover, while Aβ treatment did not affect TREM2 expression, CHF5074 significantly increased TREM2 mRNA by near 60% over the basal value. Lastly, ARG1 expression showed no change after Aβ exposition, either with or without CHF5074 addition (see FIG. 3A). None of the tested M2 mRNAs (MRC1, TREM2 and ARG1) appeared modified in cultures exposed to Aβ alone or in the presence of CHF5074 for 8 days (see FIG. 3B).

### CONCLUSIONS

The time-dependent changes in the expression profile of pro-inflammatory and anti-inflammatory microglia during exposure to Aβ and CHF5074 were analyzed. It is concluded that CHF5074 can modulate the microglial activation state in a cell-based model of Aβ-induced neuroinflammation. In mouse mixed glial culture, Aβ exposure produced an early pro-inflammatory expression of TNFα, IL-1β and iNOS mRNAs, which peaked at two days. This effect reduced with time and fell to basal level at 8 days. CHF5074 reduced pro-inflammatory genes expression at two days while it did not modify their activation at 8 days in vitro. Conversely, CHF5074 increases mRNA levels of MRC1 and TREM2, which are markers of microglia alternative activation, and appeared inhibited or unaffected by the Aβ treatment per se. These in vitro results indicate that CHF5074 can directly modulate the phenotype of active microglia, independently from interaction with circulating cells and apart from modulation of Aβ generation.

While this study was ongoing, it has been published that heterozygous loss-of-function mutations in TREM2 gene are associated with a significant increase in the risk of Alzheimer's disease (see Guerreiro et al., “TREM2 Variants in Alzheimer's Disease,” New England Journal of Medicine, Nov. 14, 2012, which is incorporated herein by reference in its entirety). This evidence has enormously emphasized the role of microglia alternative activation, and in particular of TREM2 gene, in the pathogenesis of Alzheimer's disease. TREM2 has been found to localize to microglia around plaques and neurons in the brains of TgCRND8 mice (see Guerreiro et al., “TREM2 Variants in Alzheimer's Disease,” New England Journal of Medicine, Nov. 14, 2012, which is incorporated herein by reference in its entirety). In microglia, TREM2 controls two signaling pathways to regulate the reactive phenotype of microglia. Through one pathway, TREM2 enhances phagocytosis which could be relevant to the removal of cell debris and the clearance of amyloid proteins in AD, (see Takahashi K, et al., “TREM2-trans-duced myeloid precursors mediate nervous tissue debris clearance and facilitate recovery in an animal model of multiple sclerosis, PLoS Med, 2007; 4(4):e124; Frank S, et al., “TREM2 is upregulated in amyloid plaque associated microglia in aged APP23 transgenic mice,” Glia 2008; 56:1438-47; and Piccio L, et al., “Blockade of TREM-2 exacerbates experimental autoimmune encephalomyelitis, “Eur J Immunol 2007; 37:1290-301, all of which are incorporated herein by reference in their entireties). Through the other signaling stream, TREM2 suppresses inflammatory reactivity and represses the cytokine production and secretion (see Piccio L, et al., “Blockade of TREM-2 exacerbates experimental autoimmune encephalomyelitis, “Eur J Immunol 2007; 37:1290-301, which is incorporated herein by reference in its entirety).

These results suggest that TREM2 agonists as well as drugs that modulate inflammation by switching the activation state of microglia from the M1 toward the M2 state, rather than totally repressing microglia reactivity, may be used to treat, prevent, and/or delay the onset of Alzheimer's disease.

The present results are in line with previous evidence showing the capability of CHF5074 to reduce TNFα level in cerebrospinal fluid of healthy subjects (see Imbimbo et al., “Pharmacokinetics and Pharmacodynamics of CHF5074 After Short-term Administration in Healthy Subjects,” Alzheimer Dis Assoc Disord. 2012 [Epub ahead of print] which is incorporated herein by reference in its entirety). Moreover, they suggest a wider anti-inflammatory activity of CHF5074 as modulator of microglial function, with important implications in predicting in vivo disease-modifying activity.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method for treating, preventing, and/or reducing the risk of developing Alzheimer's disease, comprising administering, to a subject in need thereof; an effective amount of a compound of formula (I): 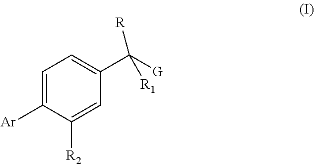
wherein:
R and R₁ are the same and are selected from the group of linear or branched C₁-C₄ alkyl; otherwise they form a 3 to 6 carbon atoms ring with the carbon atom to which they are linked;
G is: a COOR″ group wherein R″ is H, linear or branched C₁-C₄ alkyl, C₃-C₆ cycloalkyl or ascorbyl; a CONH₂ or a CONHSO₂R′″ group wherein R′″ is linear or branched C₁-C₄ alkyl or C₃-C₆ cycloalkyl; a tetrazolyl residue;
R₂ is H, CF₃, OCF₃ or a halogen selected from the group of F, Cl, Br, I, preferably fluorine. Ar is a group of formula 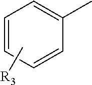
wherein R₃ represents one or more groups independently selected from:
halogen as previously defined;
—CF₃;
C₃-C₈ cycloalkyl optionally substituted with one or more C₁-C₄ alkyl and/or oxo groups;
—CH═CH₂;
—CN;
—CH₂OH;
methylendioxy or ethylendioxy;
—NO₂;
phenyl optionally substituted with one or more of the following groups:
halogen;
—CF₃;
—OCF₃;
—OH;
linear or branched C₁-C₄ alkyl;
a saturated heterocycle with at least 4 carbon atoms and at least 1 heteroatom;
C₃-C₈ cycloalkyl in turn optionally substituted with one or more of the following groups linear or branched C₁-C₄ alkyl, CF₃ or OH;
—OR₄ or —NHCOR₄ wherein R₄ is CF₃, linear or branched C₂-C₆ alkenyl or alkynyl; benzyl; phenyl optionally substituted with one or more of the following groups: halogen, CF₃, OCF₃, OH, linear or branched C₁-C₄ alkyl; a saturated heterocycle with at least 4 carbon atoms and at least 1 heteroatom; C₃-C₈ cycloalkyl in turn optionally substituted with one or more of the following groups: linear or branched C₁-C₄ alkyl, CF₃ or OH;
SR₅, SO₂R₅ or COR₅ wherein R₅ is linear or branched C₁-C₆ alkyl;
otherwise Ar is an optionally substituted heterocycle ring selected from the group of thiophene, benzothiophene, dibenzothiophene, thianthrene, pyrrole, pyrazole, furan, benzofuran, dibenzofuran, indole, isoindole, benzofurane, imidazole, benzoimidazole, oxazole, isoxazole, benzoxazole, thiazole, pyridine, pyrimidine, pyrazine, pyridazine, quinoline, isoquinoline, quinazoline, quinoxaline, cinnoline, pyrazole, pyran, benzopyran, pyrrolizine, phtalazine, 1,5-naphthyridine, 1,3-dioxole, 1,3-benzodioxole, optionally substituted with one or more groups R₃ as defined above; or a pharmaceutically acceptable salt thereof, or an ester thereof.

2. A method according to claim 1, comprising administering, to a subject in need thereof, an effective amount of compound selected from the group consisting of:
1-(2-fluoro-4′-trifluoromethylbiphenyl-4-yl)cyclopropanecarboxylic acid;
1-(2-fluoro-3′-trifluoromethoxybiphenyl-4-yl)cyclopropanecarboxylic acid;
1-(2-fluoro-4′-trifluoromethoxybiphenyl-4-yl)cyclopropanecarboxylic acid;
1-(2-fluoro-3′-trifluoromethylbiphenyl-4-yl)-cyclopropanecarboxylic acid;
1-[2-fluoro-4′-(tetrahydro-pyran-4-yloxy)-biphenyl-4-yl]-cyclopropanecarboxylic acid;
1-(2,3′,4′-trifluorobiphenyl-4-yl)cyclopropanecarboxylic acid;
1-(3′,4′-dichloro-2-fluorobiphenyl-4-yl)cyclopropanecarboxylic acid;
1-(3′-chloro-2,4′-difluorobiphenyl-4-yl)cyclopropanecarboxylic acid;
1-[2-fluoro-4′-(4-oxo-cyclohexyl)-biphenyl-4-yl]cyclopropanecarboxylic acid;
2-(2″-fluoro-4-hydroxy-[1,1′; 4′,1′]terphenyl-4″-yl)propionic acid;
1-(2,2′,4″-trifluoro[1,1′; 4′,1″]terphenyl-4-yl)cyclopropanecarboxylic acid;
1-(2′,2″-difluoro-4-hydroxy[1,1′; 4′,1″]terphenyl-4″-yl)cyclopropanecarboxylic acid;
1-(2,2′-difluoro-4″-hydroxy[1,1′; 4″,1″]terphenyl-4-yl)cyclopropanecarboxylic acid; or
a pharmaceutically acceptable salt of said compound.

3. A method according to claim 1, comprising administering, to a subject in need thereof, an effective amount of 1-(3′,4′-dichloro-2-fluorobiphenyl-4-yl)cyclopropanecarboxylic acid or a pharmaceutically acceptable salt thereof.

4. A method according to claim 1, wherein said subject has been identified as being at risk of developing Alzheimer's disease.

5. A method according to claim 1, wherein said subject has been identified as expressing a variant of TREM2.

6. A method according to claim 1, wherein said subject has been identified as expressing the R47H variant of TREM2.

7. A method according to claim 1, wherein said subject has been identified as expressing a variant of ABCA7, MS4A6A/MS4A4E, EPHA1, CD33, or CD2AP.

8. A method according to claim 1, wherein said subject has been identified as expressing a variant of CD33.

9. A method for treating, preventing, and/or reducing the risk of developing multiple sclerosis, comprising administering, to a subject in need thereof, an effective amount of a compound of formula (I): 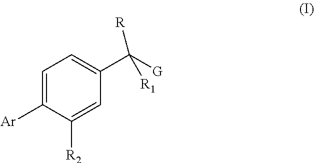
wherein:
R and R₁ are the same and are selected from the group of linear or branched C₁-C₄ alkyl; otherwise they form a 3 to 6 carbon atoms ring with the carbon atom to which they are linked;
G is: a COOR″ group wherein R″ is H, linear or branched C₁-C₄ alkyl, C₃-C₆ cycloalkyl or ascorbyl; a CONH₂ or a CONHSO₂R′″ group wherein R′″ is linear or branched C₁-C₄ alkyl or C₃-C₆ cycloalkyl; a tetrazolyl residue;
R₂ is H, CF₃, OCF₃ or a halogen selected from the group of F, Cl, Br, I, preferably fluorine. Ar is a group of formula 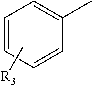
wherein R₃ represents one or more groups independently selected from:
halogen as previously defined;
—CF₃;
C₃-C₈ cycloalkyl optionally substituted with one or more C₁-C₄ alkyl and/or oxo groups;
—CH═CH₂;
—CN;
—CH₂OH;
methylendioxy or ethylendioxy;
—NO₂;
phenyl optionally substituted with one or more of the following groups:
halogen;
—CF₃;
—OCF₃;
—OH;
linear or branched C₁-C₄ alkyl;
a saturated heterocycle with at least 4 carbon atoms and at least 1 heteroatom;
C₃-C₈ cycloalkyl in turn optionally substituted with one or more of the following groups linear or branched C₁-C₄ alkyl, CF₃ or OH;
—OR₄ or —NHCOR₄ wherein R₄ is CF₃, linear or branched C₂-C₆ alkenyl or alkynyl; benzyl; phenyl optionally substituted with one or more of the following groups: halogen, CF₃, OCF₃, OH, linear or branched C₁-C₄ alkyl; a saturated heterocycle with at least 4 carbon atoms and at least 1 heteroatom; C₃-C₈ cycloalkyl in turn optionally substituted with one or more of the following groups: linear or branched C₁-C₄ alkyl, CF₃ or OH;
SR₅, SO₂R₅ or COR₅ wherein R₅ is linear or branched C₁-C₆ alkyl;
otherwise Ar is an optionally substituted heterocycle ring selected from the group of thiophene, benzothiophene, dibenzothiophene, thianthrene, pyrrole, pyrazole, furan, benzofuran, dibenzofuran, indole, isoindole, benzofurane, imidazole, benzoimidazole, oxazole, isoxazole, benzoxazole, thiazole, pyridine, pyrimidine, pyrazine, pyridazine, quinoline, isoquinoline, quinazoline, quinoxaline, cinnoline, pyrazole, pyran, benzopyran, pyrrolizine, phtalazine, 1,5-naphthyridine, 1,3-dioxole, 1,3-benzodioxole, optionally substituted with one or more groups R₃ as defined above; or a pharmaceutically acceptable salt thereof or an esters thereof.

10. A method according to claim 9, comprising administering, to a subject in need thereof, an effective amount of compound selected from the group consisting of:
1-(2-fluoro-4′-trifluoromethylbiphenyl-4-yl)cyclopropanecarboxylic acid;
1-(2-fluoro-3′-trifluoromethoxybiphenyl-4-yl)cyclopropanecarboxylic acid;
1-(2-fluoro-4′-trifluoromethoxybiphenyl-4-yl)cyclopropanecarboxylic acid;
1-(2-fluoro-3′-trifluoromethylbiphenyl-4-yl)-cyclopropanecarboxylic acid;
1-[2-fluoro-4′-(tetrahydro-pyran-4-yloxy)-biphenyl-4-yl]-cyclopropanecarboxylic acid;
1-(2,3′,4′-trifluorobiphenyl-4-yl)cyclopropanecarboxylic acid;
1-(3′,4′-dichloro-2-fluorobiphenyl-4-yl)cyclopropanecarboxylic acid;
1-(3′-chloro-2,4′-difluorobiphenyl-4-yl)cyclopropanecarboxylic acid;
1-[2-fluoro-4′-(4-oxo-cyclohexyl)-biphenyl-4-yl]cyclopropanecarboxylic acid;
2-(2″-fluoro-4-hydroxy-[1,1′; 4′,1′]terphenyl-4″-yl)propionic acid;
1-(2,2′,4″-trifluoro[1,1′;4′,1″]terphenyl-4-yl)cyclopropanecarboxylic acid;
1-(2′,2″-difluoro-4-hydroxy[1,1′;4′,1″]terphenyl-4″-yl)cyclopropanecarboxylic acid;
1-(2,2′-difluoro-4″-hydroxy[1,1′; 4″,1″]terphenyl-4-yl)cyclopropanecarboxylic acid; or
a pharmaceutically acceptable sal of said compound.

11. A method according to claim 9, comprising administering, to a subject in need thereof, an effective amount of 1-(3′,4′-dichloro-2-fluorobiphenyl-4-yl)cyclopropanecarboxylic acid or a pharmaceutically acceptable salt thereof.

12. A method according to claim 9, wherein said subject has been identified as expressing a variant of TREM2.

13. A method according to claim 9, wherein said subject has been identified as expressing the R47H variant of TREM2.

14. A method according to claim 9, wherein said subject has been identified as expressing a variant of ABCA7, MS4A6A/MS4A4E, EPHA1, CD33, or CD2AP.

15. A method according to claim 9, wherein said subject has been identified as expressing a variant of CD33.
